(19) United States
(12) Patent Application Publication
(10) Pub. No.: US 2022/0151365 A1
(43) Pub. Date: May 19, 2022

(54) Device in the form of a composition felt pen to instantly color the white hairs of the beard and hair.

(71) Applicant: Cédric désiré SMITH, Paris (FR)

(72) Inventor: Cédric désiré SMITH, Paris (FR)

(21) Appl. No.: 16/640,989

(22) PCT Filed: Jun. 13, 2019
(86) PCT No.: PCT/IB2019/054958

§ 371 Date: Mar. 11, 2021

(51) Int. Cl. A45D 19/00; A45D 19/02; A61K 8/02; A61K 8/34; A61K 8/92; A61Q 5/06; A45D 27/00

(52) U.S. Cl. CPC A45D 19/0083; A45D 19/02; A61K 8/0208; A61K 8/0241; A45D 2034/005; A61K 8/922; A61Q 5/065; A45D 27/00; A61K 8/34

(57) ABSTRACT

The invention relates to a device in the form of a composition felt pen for quickly and instantaneously self-coloring the sparse white hairs of the beard and white hair. It consists of a body that encloses a porous reservoir impregnated with a cartridge of coloring composition and connects to a tip of synthetic fiber. This is a real novelty for men.

Device in the form of a composition felt-tip pen to instantly color the white hairs of the beard and hair.

The invention is in the field of cosmetics, preferably for men. The invention relates to a device in the form of a composition felt-tip pen to instantly color the white hair of the heard and hair. The felt-tip pen comprises a coloring composition for applying it to the white hairs of the beard and the white hair preferably runs. The coloring composition is applied using the device or all coloring components are mixed, ready for use. This device is in the form of a felt-tip pen, it is constituted like a felt-tip marker pen. It is a felt pen whose body contains a porous reservoir at the end impregnated with a staining cartridge and connects to a tip made of synthetic material (wide tip of 2 mm to 4 mm and 1 cm to 1.5 cm long), also impregnated which allows a light and. comfortable spreading. The coloring composition flows by capillarity and impregnates the point which is porous. Originally, this tip was in natural felt, which explains the name of this pen. The method of application of the coloration characterized in that is applied on the white hair of the beard and white hair rather short from the root to the tip, the composition with the aid of the felt leaning thereof. This device the dye pen has the body which differs in length and width (between 7 cm and 15 cm in length and 1 cm to 1.5 cm in diameter) and comes in different materials like plastic, glass, wood, metal, recycled materials, gold or silver for a more luxurious version.

The dye felt pen is easy to use industrially and can be made in large numbers at most cosmetic factories around the world . . . This application device is “the dyestuff pen” is a technical answer to colorations. This device is in the form of a felt-tip pen to color even without risk and quickly the unsightly white hairs of the beard and the short and sparse white hair of men. This novelty is useful and essential to cosmetics and all men regardless of their ethnicity dice the appearance of the first white hairs. Device in the form of a felt-tip pen that can be used several times for the application of a composition for coloring the white and sparse hairs of the beard and the hair. The composition comprising water, an alcohol, at least 15% by weight relative to the weight of the fine particle composition of a natural plant pigment at least one vegetable Shea or coconut butter and/or a vegetable oil. All coloring components of the composition being mixed and ready for use, characterized in that the marker pen contains a porous reservoir which consists of a coloring cartridge comprising the composition, connected to the porous and impregnated synthetic fiber tip of the coloring composition.

A method of applying coloring, characterized in that the composition is applied to the white hairs of the beard and the hair from the root to the tip using the felt pen of any one of claims 1 to 3, tilting the felt-tip pen. “The Color Felt Pen” is a technical answer to the coloring of the beard and white hair. The application process is very simple. Easy to use, it offers an exclusive ease of application that is a real novelty for men and for cosmetics. The dye Felt pen is easy to industrial application with Benefits, a beautifying and rejuvenating effect, waterproof for 24 h, Quick and easy application, small and discreet with the possibility of having it always with several shades of color possible blond, brown, chestnut, red. This is a real novelty for men and is a real opportunity for women.

Currently, for beards and hair there are shampoos or dyes difficult to apply requiring in most cases the intervention of a third party or a hairdresser. The current technique consists of a dyeing that must be done in several steps: mix products, apply the color, distribute the product, leave to ask, rinse several times, and dry. Now the coloring felt-tip pen is revolutionizing the current technique. Now, in two minutes, the white hairs of the beard and the rather short white hair are simply colored from root to tip. Easy to use, it offers an exclusive ease of application that is a real novelty for men and is welcome for women.

The composition for coloring the white hairs of the beard and white hair preferably short which comprises: water, an alcohol, at least 15% by weight based on the weight of the composition of fine particles of a natural plant pigment at least one shea butter or coconut vegetable butter and/or a sweet almond oil is a so-called natural plant coloring which is composed of plant extracts containing natural pigments with high coloring power. This natural coloring neither is without chemical dye or perfume, nor oxidizes nor ammonia, consequently does it not cause any polluting discharges. Particularly soft it does not attack the structure of the hair of the beard and is composed at 100°/° of vegetable ingredients. This coloration exploits the natural dyes of dyeing plants, used for hundreds of years to dye; there is henna, madder, blueberry, beetroot, rhubarb, and coffee. This plant color coats the keratin fiber and forms a protective sheath around it. The pigments of the plant are fixed only on the outer layers of the hair of the beard and as the color does not penetrate the inside of the hair unlike the chemical dye, it fades gradually without demarcation.

Free of toxic substances, it respects the ecosystem of the hairs of the beard. It does not oxidize them, does not change their chemical structure and stays on the surface of the fiber. This composition is very well tolerated; the risks of allergic reactions are almost zero. White beard hair and very weak or damaged white hair can use it. The natural pigments of the color are available in several shades such as brown, chestnut, blond, and auburn and red. The brown shades are formulated with madder, indigo, walnut, chicory, sandalwood . . . The blond shades with extracts of cornflower, rhubarb, chamomile, and centaury. The red hues with extracts of tea, hibiscus, boxwood, and buckthorn. The color also include composition of hyaluronique acid and thus become a color care. Hyaluronique acid is a naturally occurring carbohydrate found in the human body. This hyaluronique acid is obtained from natural fermentation and is generally in pure form at more than 90°/°. Hyaluronique acid helps strengthen the hair of the beard. The white hairs of the beard and the very fragile or damaged short white hair can use

1) Device in the form of a felt-tip pen which can be used several times for the application of a composition for coloring the white and sparse hairs of the beard and of the half.
The composition comprising
a) Water
b) An alcohol
c) At least 15% of the weight of the tine particle composition of a natural plant pigment
d) At least one Shea or coconut vegetable butter and/or a vegetable oil.
All coloring components of the composition being mixed and ready for use, characterized in that the marker pen contains a porous reservoir which consists of a coloring cartridge comprising the composition, connected to the porous and impregnated synthetic fiber tip of the coloring composition.

2) Device according to claim 1 characterized in that the felt pen has the body which differs in length and width between (7 cm and 15 cm in length and 1 cm and 1.5 cm in diameter) and the tip up to 1.5 cm of length.

3) Device according to claim 1 or 2 characterized in that the body of the felt pen is declined in different materials such as plastic, glass, wood, metal, recycled materials, gold or silver.

4) Method of applying the coloring, characterized in that the felt-tip pen is applied to the white hairs of the beard and the hairs from root to tip the composition using the felt pen according to one of claims 1 to 3, by tilting the felt-tip pen.
